(19) United States
(12) Patent Application Publication
(10) Pub. No.: US 2023/0133214 A1
(43) Pub. Date: May 4, 2023

(54) COMPOSITE GUIDE PLATE

(71) Applicant: ZUGA MEDICAL CHENGDU CO., LTD, CHENGDU (CN)

(72) Inventors: CHAN Q. WANG, CHAGRIN FALLS, OH (US); ZHONGYU YU, CHENGDU (CN); YUE LI, CHENGDU (CN)

(73) Assignee: ZUGA MEDICAL CHENGDU CO., LTD, CHENGDU (CN)

(21) Appl. No.: 17/625,050

(22) PCT Filed: Jun. 24, 2020
(86) PCT No.: PCT/CN2020/097996

§ 371 Date: Jan. 5, 2022

(30) Foreign Application Priority Data
Jul. 5, 2019 (CN) 201921044491.4

(51) Int. Cl. A61C 1/08; B33Y 80/00

(52) U.S. Cl. CPC A61C 1/084; B33Y 80/00; A61B 6/14

(57) ABSTRACT

The present utility model relates to a composite guide plate, comprising a base guide plate (1) and an implant guide plate (7), or comprising a base guide plate (1) and a repair guide plate (10), or comprising a base guide plate (1), an implant guide plate (7) and a repair guide plate (10), wherein the base guide plate (1) is provided with a retention pin sleeve (2); the implant guide plate (7) is provided with an implant sleeve (8) and a retention pin sleeve fastener (5); the retention pin sleeve fastener (5) is fastened on the retention pin sleeve (2) to connect the implant guide plate (7) to the base guide plate (1); the repair guide plate (10) is provided with the retention pin sleeve fastener (5); and the retention pin sleeve fastener (5) is fastened on the retention pin sleeve (2) to connect the repair guide plate (10) to the base guide plate (1). By means of the composite guide plate provided in the present utility model, the defect of traditional methods whereby the repeated removal and fitting of retention pins and guide plates is required can be overcome. Moreover, all the guide plates can be printed with resin, thereby greatly reducing clinical medical costs and the medical costs of processing plants.

### TECHNICAL FIELD

The utility model claims a composite guide plate, which can be applied to oral planting, repairing and so on need preoperative planning, accurately predicting the case of surgical result, belonging to the oral medical field.

### TECHNICAL BACKGROUND

Existing surgical guide plate is used for planting after the jaw osteotomy, the doctor generally needs to firstly design a bone-cutting guide plate containing fixing nail sleeve, then designing a planting guide plate containing fixing nail sleeve and planting sleeve. The doctor needs to take off the osteotomy guide plate after wearing the osteotomy guide plate, wearing the planting guide plate, and then performing the planting operation.

The defects of the traditional scheme are as follows: In the manufacturing process, the preoperative design, manufacturing work is complicated, many processes need to repeat the same operation; the doctor has a lot of time waste on picking and fixing nail; on the guide plate, the efficiency is low; the patient waiting for the designed period is long and operation time is long. In addition, the traditional guide cannot plan the position of the prosthesis, generally after finishing the planting operation and then manufacturing temporary repairing body, increasing the diagnosis time of the patient.

At present, the latest technology, such as the Guide Smile guide plate made by ROE Lab, integrates the osteotomy, planting and repairing together. Specifically, comprising the following structure: osteotomy guide plate of metal printing and planting guide plate, wherein the osteotomy guide plate is fixedly located at the jaw bone through the fixing nail; the planting guide plate is tightly connected with the osteotomy guide plate through the Swiss lock; the repairing guide plate is connected with the osteotomy guide plate by repairing the substrate guide plate; the repairing substrate guide plate is also connected with the osteotomy guide plate through the Swiss lock. The technology solves the problem that the traditional technology in anti-repeated area fixing nail and cannot plan the repairing body in advance, but the connection mode of the Swiss lock causes the guide must be metal printing to ensure the strength and connection precision, increasing the manufacturing cost.

The content of the utility model

The utility model solves the shortage of the traditional technology and manufacturing cost of the problem, providing a composite guide plate, the osteotomy, planting and repairing on a guide plate sleeve can be realized. optimizing the design process, no need to respectively manufacture substrate (osteotomy) guide plate, planting guide plate, so as to overcome the traditional method needs to repeatedly take the defect of the fixing nail and guide plate; at the same time, due to the substrate guide plate; the planting guide plate and repairing guide plate are respectively provided with a locating plane reinforcing support strength, and buckled by the fixing nail sleeve and fixing nail sleeve buckle; therefore, all guide plates can be printed by resin, which greatly reduces the medical cost of clinical and processing factory; at the same time, it ensures the operation precision; The patient's diagnosis experience has also risen by a level.

In order to solve the above technical problem, the utility model adopts the following solution:

(1) a composite guide plate, a substrate guide plate and a planting guide plate; or a substrate guide plate and a repairing guide plate; or a substrate guide plate, a planting guide plate and a repairing guide plate; a fixing nail sleeve is arranged on the substrate guide plate; When using, the fixing nail is punched on the fixing nail sleeve; the substrate guide plate is fixed; an implant sleeve and a retaining nail sleeve buckle are arranged on the planting guide plate; the fixing nail sleeve is buckled on the fixing nail sleeve so that the planting guide plate is connected with the substrate guide plate; a fixing nail sleeve buckle is arranged on the repairing guide plate; the fixing nail sleeve is buckled on the fixing nail sleeve so that the repairing guide plate is connected with the substrate guide plate.

(2) according to (1) the composite guide plate, further provided with a locating plane on the substrate guide plate; the planting guide plate is further provided with a tongue side locating plane; the tongue side locating plane is matched with the locating plane of the substrate guide plate; the repairing guide plate is further provided with a tongue side locating plane; the tongue side locating plane is matched with the locating plane of the substrate guide plate.

(3) according to (1) or (2) the composite guide plate, further comprising a positioning guide plate, the positioning guide plate is provided with a fixing nail sleeve buckle and a tongue side locating plane; the tongue side locating plane is matched with the locating plane of the substrate guide plate; the fixing nail sleeve is buckled on the fixing nail sleeve so that the positioning guide plate is connected with the substrate guide plate.

(4) according to (1)-(3) any one of the composite guide plate; the planting guide plate, repairing guide plate and guide plate are respectively provided with a connecting rod; the connecting rod is connected with the fixing nail sleeve buckle.

(5) according to (1)-(4) any one of the composite guide plate; the repairing guide plate is provided with a repairing body; the repairing body is connected with the tongue side locating plane and fixing nail sleeve buckle.

(6) according to (1)-(5) any one of the composite guide plate, when the repairing body is finally planted, grinding the tongue side locating plane, fixing nail sleeve buckle and connecting rod.

(7) according to (1)-(6) any one of the composite guide plate, further comprising an implant, implant implant through implant, the implant is connected with a temporary base.

(8) according to (1)-(7) any one of the composite guide plate, the temporary abutment and repairing body on the repairing guide plate.

(9) according to (1)-(8) any one of the composite guide plate; the inner surface of the fixing nail sleeve buckle is attached to the outer surface of the fixing nail sleeve; the radian of the fixing nail sleeve buckle is 180 degrees to −240 degrees; the outer surface of the fixing nail sleeve buckle is concave-convex structure, embedding structure, square and rectangular structure, hexagonal structure, diamond-shaped structure or circular arc structure.

(10) The composite guide plate according to any one of the (1) to (9), substrate guide plate, planting guide plate, repairing guide plate and material of the guide plate is resin.

The utility model at least comprises the following beneficial effects:

1. The composite guide plate provided by this utility model, no need to respectively manufacture substrate (osteotomy) guide plate, planting guide plate, can reduce greatly shorten the manufacturing period, simplify the doctor clinical operation procedure, reduce the patient waiting time, reduce the clinical medical cost, greatly improve the patient diagnosis experience;

2nd, according to the bone plane height provided by the composite guide plate, width information, implant implanting orientation information and repair the occlusion information of the dentists, the doctor can refer to the osteotomy plane indicated by the substrate guide plate for osteotomy in the operation, referring to the depth indicated by the planting guide plate; axial and so on; according to the engagement information provided by the repairing guide plate for bonding the repairing body and temporary abutment, finally finishing planting and repairing; compared with the traditional manual operation, it not only saves time, saves trouble, saves labour, but also greatly improves the operation precision;

3rd, compared with the Guide Smile guide plate of Chrome, the utility model directly buckles the fixing nail sleeve buckle on the fixing nail sleeve; it does not need to make redundant accessories (such as Swiss lock and so on); it is convenient for the clinical operation of the doctor; at the same time, because the substrate guide plate, planting guide plate and repairing guide plate are respectively provided with a locating plane reinforcing support strength; the utility model has no limit of material; under the premise of ensuring the precision of the guide plate, it can use resin, peek material with low cost, which saves a lot of cost for the doctor and patient.

The other advantages of the utility model, the target and features will be reflected by the following description, part will be understood by those skilled in the art by the research and practice of the utility model.

The related term meaning of this utility model is as follows:

1. CBCT Cone Beam Computing Tomography): is a cone-beam projector computer recombination fault image device for obtaining oral three-dimensional image data information.

2. CBCT data refers to the jaw bone obtained by CBCT, tooth body and other related information.

(3. STL Triangulation Language): for representing a file format of the triangular grid. Its file format is very simple, and it is widely used.

4. STL data refers to the information related to the dental tissue, soft tissue and so on the oral cavity. it can use high precision oral surface scanning device to perform fine scanning of oral tissue surface, obtaining STL data containing dental tissue, soft tissue and other related information.

5. The osteotomy guide plate: a guide plate for cutting the sharp or uneven part of the crest of the alveolar ridge, and then inserting the planting guide plate part, realizing the preparation of the planting hole.

6. The mucous membrane supporting type guide plate: It is used for treating the guide plate without tooth jaw. The main advantage is that it can provide better operation view, better control the planting depth. However, this type of guide plate due to the ability of the mucous membrane, if there is no retention nail auxiliary retention will be displaced.

7. The tooth support type guide plate: Single or more implant cases are performed using a dental support guide. The use of a guide plate can be used to achieve stability in a surgical procedure suitable for free end cases (which can be limited).

8. The Bone Support Guide: The invention claims a guide plate supported and fixed by jaw bone.

(9. DICOM Imaging and Communications in Medicine): Medical digital image transmission protocol is a group of universal standard protocol, for medical image processing, storage, printing and transmission. It contains definition of file format and network communication protocol, based on TCP/IP, and contacts each system by TCP/IP, and can integrate medical image instrument of different manufacturers, server, work station, printer and network device.

10. Stack@fixing nail: The invention is mainly used for oral non-tooth maxillofacial implant operation, so as to avoid the guide plate shift caused by the transmutability of the mucosa.

The following are the following: divided into a fixing nail sleeve and an implant sleeve, for guiding the clinician to accurately implant the retention nail or implant according to the designed position of the retention nail or implant.

### DESCRIPTION OF PICTURES

The utility model will be further described in detail with reference to the accompanying drawings and specific embodiments.

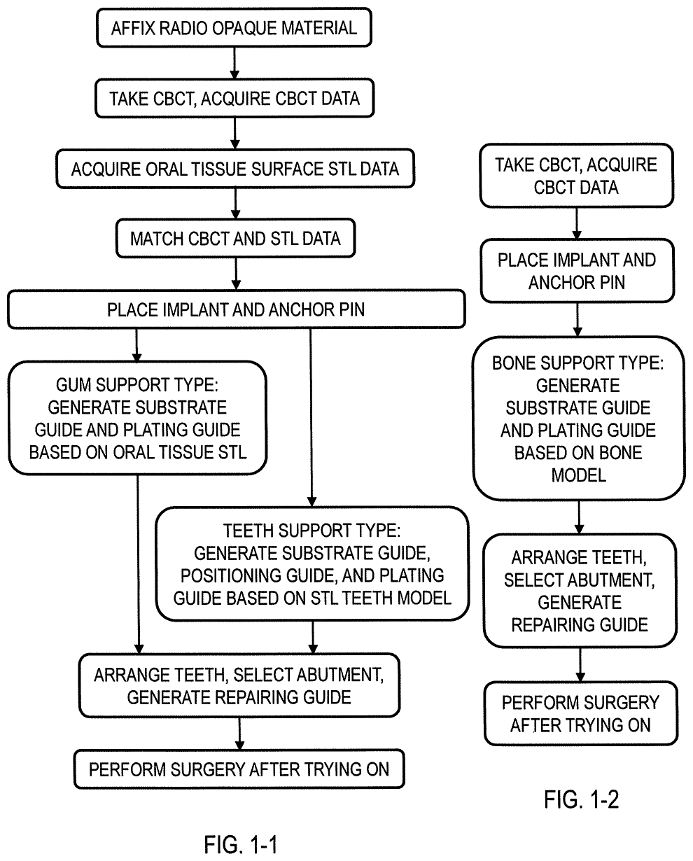

FIG. 1-1 is a mucosal support type and tooth support type composite guide plate generating and using flow chart;

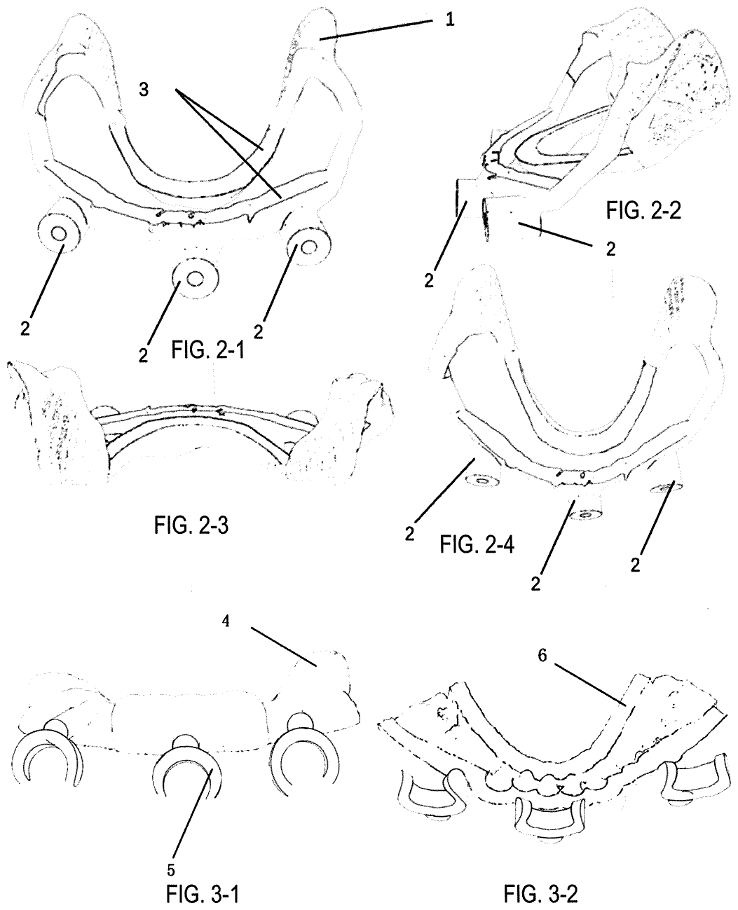

FIG. 1-2 is a bone support type composite guide plate generating and using flow chart;

FIG. 2-1 is a front view of the substrate guide plate;

FIG. 2-2 is a right view of the substrate guide plate;

FIG. 2-3 is a rear view of the substrate guide;

FIG. 2-4 is a top view of the substrate guide;

FIG. 3-1 is a schematic diagram of the guide plate;

FIG. 3-2 is a bottom view of FIG. 3-1;

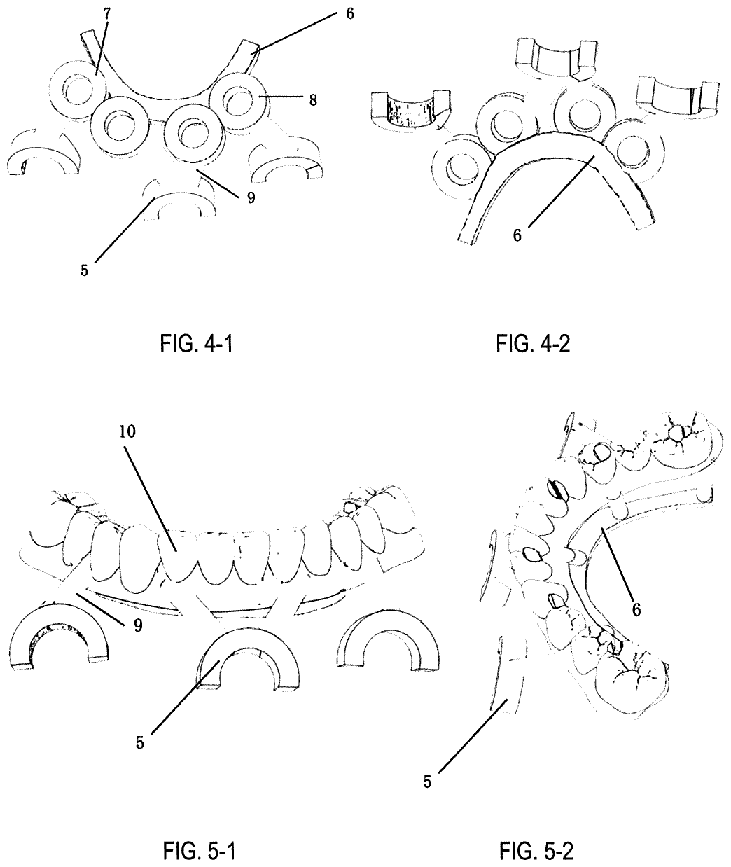

FIG. 4-1 is a schematic diagram of the planting guide plate;

FIG. 4-2 is a bottom view of FIG. 4-1;

FIG. 5-1 the schematic diagram of repairing guide plate;

FIG. 5-2 the three-dimensional graph of repairing guide plate;

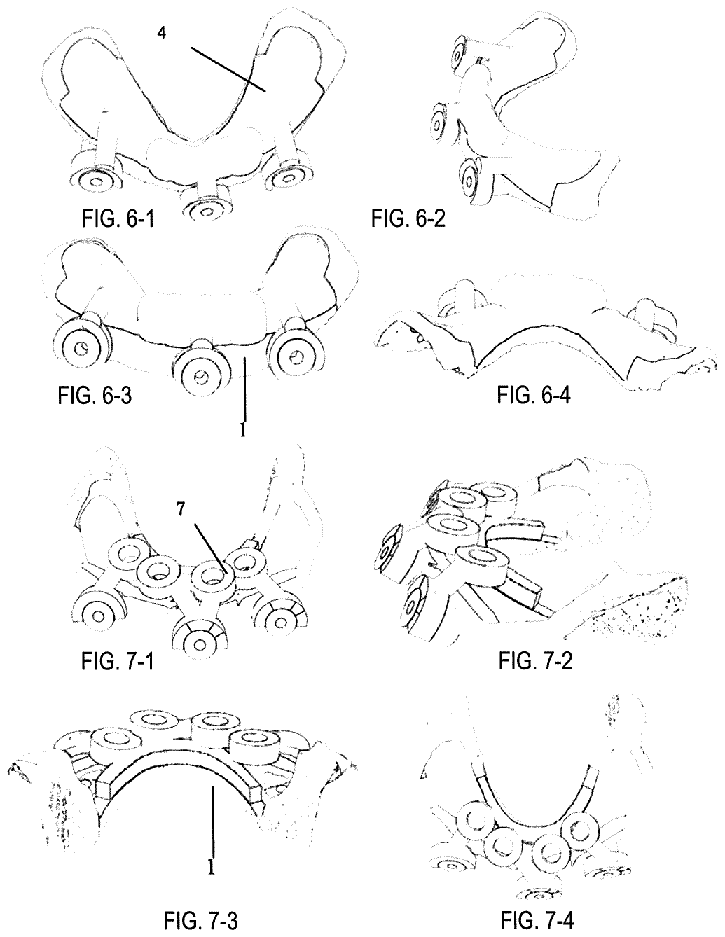

FIG. 6-1 is a positioning guide plate matched with the top view of the substrate guide;

FIG. 6-2 is a right view of the positioning guide plate matched with the substrate guide plate;

FIG. 6-3 is a front view of the positioning guide plate matched with the substrate guide plate;

FIG. 6-4 is a rear view of the positioning guide plate matched with the substrate guide plate;

FIG. 7-1 is a planting guide plate matched with the main view of the substrate guide plate;

FIG. 7-2 is a planting guide plate matched with the right view of the substrate guide plate;

FIG. 7-3 is a planting guide plate matched with the rear view of the substrate guide;

FIG. 7-4 is a planting guide plate matched with the top view of the substrate guide;

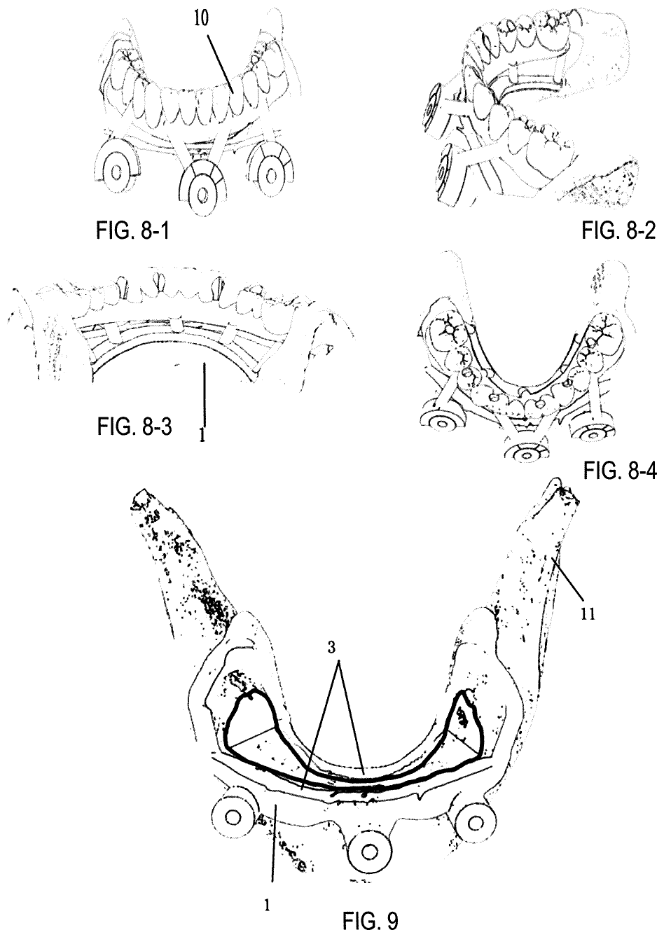

FIG. 8-1 is a repair guide matched with the main view of the substrate guide plate;

FIG. 8-2 is a repairing guide plate matched with the three-dimensional pattern of the substrate guide plate;

FIG. 8-3 is a repair guide matched with the rear view of the substrate guide plate;

FIG. 8-4 is a view of the repairing guide plate matched with the substrate guide plate;

FIG. 9 is a schematic view of the osteotomy after wearing the substrate guide plate;

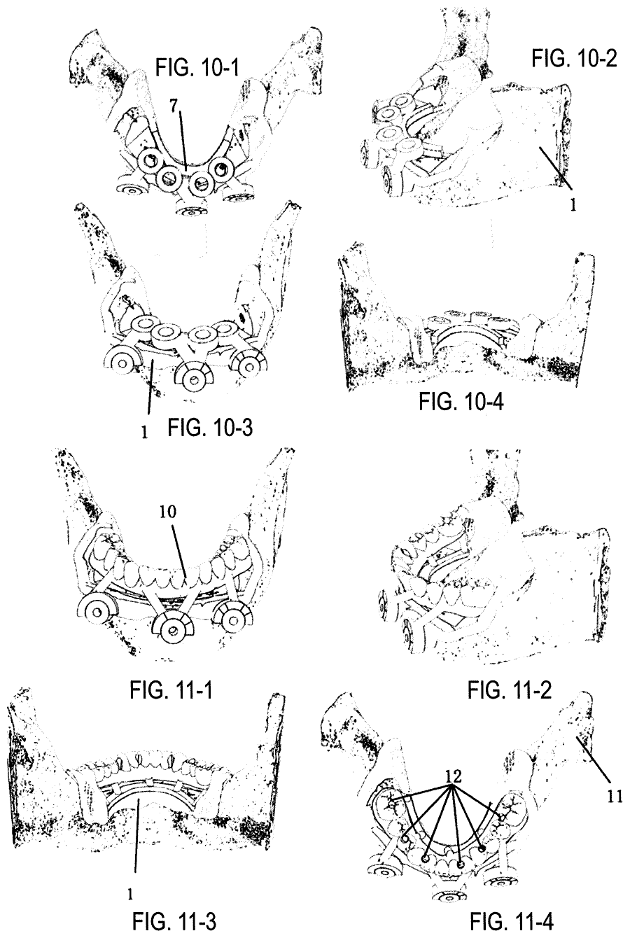

FIG. 10-1 is a planform after the planting guide plate matched with the substrate guide plate;

FIG. 10-2 is a bone after the planting guide plate matched with the right view of the substrate guide plate;

FIG. 10-3 is a bone after the planting guide plate matched with the front view of the substrate guide plate;

FIG. 10-4 is a rear view after the osteotomy guide plate matched with the substrate guide plate;

FIG. 11-1 is a repair guide located on the main view of the substrate guide plate;

FIG. 11-2 is a repair guide located on the right view of the substrate guide plate;

FIG. 11-3 is a repair guide located on the back view of the substrate guide;

FIG. 11-4 is a view of the repairing guide plate located on the substrate guide;

In the picture, 1 is a base guide plate, 2 is a fixing nail sleeve, 3 is a locating plane, 4 is a locating guide plate, 5 is fixing nail sleeve buckle, 6 is tongue side locating plane, 7 is a planting guide plate, 8 is an implant sleeve, 9 is a connecting rod, 10 is repairing guide plate, 11 is jaw bone, 12 is a temporary base.

### Specific Implementation Examples EXAMPLE 1

As shown in FIGS. 2-1 to 2-4 3-2, FIGS. 3-1 4-1 to 4-2 5-2, picture 5-1 4-2 to 5-2, belonging to the tooth support composite guide plate, namely suitable for the patient and tooth condition (using the non-flap of the push-to-pull type case), comprising a substrate guide plate 1, guide plate 4; planting guide plate 7 and repairing guide plate 10.

a fixing nail sleeve 2 and a locating plane 3 are arranged on the substrate guide plate 1; When using, fixing nail is driven on the fixing nail sleeve 2; the substrate guide plate 1 is fixed.

the locating guide plate 4 is provided with a fixing nail sleeve buckle 5 and a tongue side locating plane 6; the tongue side positioning plane 6 is matched with the positioning plane 3 of the substrate guide plate 1; the fixing nail sleeve buckle 5 is buckled on the fixing nail sleeve 2 so that the locating guide plate 4 is connected with the substrate guide plate 1 (as shown in FIGS. 6-1 to 6-4).

the planting guide 7 is provided with an implant sleeve 8; the fixing nail sleeve buckle 5 and tongue side locating plane 6; the fixing nail sleeve buckle 5 is buckled on the fixing nail sleeve 2 so that the planting guide plate 7 is connected with the substrate guide plate 1; The tongue side positioning plane 6 cooperates with the positioning plane 3 of the base guide plate 1 (as shown in FIGS. 7-1 to 7-4).

the repairing guide plate 10 is provided with a fixing nail sleeve buckle 5 and a tongue side locating plane 6; the fixing nail sleeve buckle 5 is buckled on the fixing nail sleeve 2 so that the repairing guide plate 10 is connected with the substrate guide plate 1; The tongue side positioning plane 6 cooperates with the positioning plane 3 of the base guide plate 1 (as shown in FIGS. 8-1 to 8-4). the repairing guide plate 10 is provided with a repairing body; the repairing body is connected with the tongue side locating plane 6 and fixing nail sleeve buckle 5.

the planting guide plate 7, repairing guide plate 10 and guide plate 4 are respectively provided with a connecting rod 9; the connecting rod 9 is connected with the fixing nail sleeve buckle 5.

In use, implant is implanted through implant sleeve 8, on the implant is connected with temporary base 12.

The temporary abutment 12 is bonded to the prosthesis on the repair guide plate 10 (as shown in FIG. 11-4

the inner surface of the fixing nail sleeve buckle 5 is attached to the outer surface of the fixing nail sleeve 2; the radian of the fixing nail sleeve buckle 5 is 180 degrees to −240 degrees; the outer surface of the fixing nail sleeve buckle 5 is a concave-convex structure, a embedding structure, a square and a rectangular structure, a hexagonal structure, a rhombic structure or a circular arc structure.

when the repairing body is finally planted, grinding the tongue side locating plane 6, fixing nail sleeve buckle 5 and connecting rod 9.

substrate guide plate 1, planting guide 7, repairing guide plate 10 and guide plate 4 is made of resin.

### EMBODIMENT 2

As shown in FIGS. 2-1 to 2-4, FIGS. 5-1 to 5-2, FIGS. 5-1 to 5-2, a composite guide plate, suitable for mucosal support type composite guide plate and a bone support type composite guide plate, namely suitable for the patient without tooth condition (wherein the bone support type composite guide plate needs osteotomy), comprising a substrate guide plate 1; planting guide plate 7 and repairing guide plate 10. The difference between this embodiment and embodiment 1 is that it does not comprise a positioning guide plate.

a fixing nail sleeve 2 and a locating plane 3 are arranged on the substrate guide plate 1; When using, fixing nail is driven on the fixing nail sleeve 2; the substrate guide plate 1 is fixed.

the planting guide 7 is provided with an implant sleeve 8; the fixing nail sleeve buckle 5 and tongue side locating plane 6; the fixing nail sleeve buckle 5 is buckled on the fixing nail sleeve 2 so that the planting guide plate 7 is connected with the substrate guide plate 1; The tongue side positioning plane 6 cooperates with the positioning plane 3 of the base guide plate 1 (as shown in FIGS. 7-1 to 7-4).

the repairing guide plate 10 is provided with a fixing nail sleeve buckle 5 and a tongue side locating plane 6; the fixing nail sleeve buckle 5 is buckled on the fixing nail sleeve 2 so that the repairing guide plate 10 is connected with the substrate guide plate 1; The tongue side positioning plane 6 cooperates with the positioning plane 3 of the base guide plate 1 (as shown in FIGS. 8-1 to 8-4). the repairing guide plate 10 is provided with a repairing body; the repairing body is connected with the tongue side locating plane 6 and fixing nail sleeve buckle 5.

the planting guide plate 7 and repairing guide plate 10 are respectively provided with a connecting rod 9; the connecting rod 9 is connected with the fixing nail sleeve buckle 5.

In use, implant is implanted through implant sleeve 8, on the implant is connected with temporary base 12.

The temporary abutment 12 is bonded to the prosthesis on the repair guide plate 10 (as shown in FIG. 11-4

the inner surface of the fixing nail sleeve buckle 5 is attached to the outer surface of the fixing nail sleeve 2; the radian of the fixing nail sleeve buckle 5 is 180 degrees to −240 degrees; the outer surface of the fixing nail sleeve buckle 5 is a concave-convex structure, a embedding structure, a square and a rectangular structure, a hexagonal structure, a rhombic structure or a circular arc structure.

when the repairing body is finally planted, grinding the tongue side locating plane 6, fixing nail sleeve buckle 5 and connecting rod 9.

the substrate guide plate 1, planting guide 7 and repairing guide plate 10 is made of resin.

### EXAMPLE 3

A composite guide plate, belonging to the dental support type composite guide plate, wherein the preparation and use (using the non-flap of the push-to-pull type case) process is as follows (as shown in FIG. 1-1):

1st, the barrier material is adhered on the tooth; the barrier material is adhered on the gingival mucous membrane or/and barrier material is adhered on other anatomical features; on the tooth, gingival mucosa or/and other anatomical features at least 4 point adhesive resistance material; Other anatomical features include, mucosal turning, bone protrusion, tongue protuberance.

2nd, carrying out CBCT shooting; obtaining CBCT data with anti-reflection material information.

3rd, obtaining the oral surface STL data with impedance material information, the method comprising:

a) taking the oral cavity impression; the blocking material will leave the mark on the corresponding position of the impression material; after the mould is hard to be printed, copying the blocking material to the impression; using the oral surface scanning device to scan the oral impression; obtaining the STL data with the information of the blocking material;

b) or using oral surface scanning device to directly scan in the oral cavity; obtaining STL data with anti-reflection material information;

c) or bonding the barrier material at the thickest and thinnest part of the oral mucosa, then displaying the bone in the CBCT data according to CBCT; tooth information and suspended resistance material information; because the suspension of the impedance material is adhered on the soft tissue, by analyzing the impedance material and bone relation, it can analyze the relationship and thickness of the bone and mucosa, so as to obtain soft tissue information, finally obtaining STL data.

4. matching the CBCT data with the STL data: integrating the CBCT data and STL data, taking the information of the blocking material as the matching basis, by adjusting the tooth tissue information in the CBCT data and STL data, finally matching the jaw bone information, tooth tissue information, soft tissue information.

5. Planning and retention of the implants and retention pins: according to the CBCT data and STL data matching information, obtaining the pre-tooth position and jaw bone condition, then according to the pre-tooth position and jaw bone condition, planning implant and fixing nail position.

The substrate guide plate 1 is formed by the following steps: designing the fixing nail sleeve 2 and locating plane 3, the combination is a whole, to obtain the base guide plate 1 with a fixing nail sleeve (as shown in FIGS. 2-1 to 2-4).

The positioning guide plate 4 is formed by the following steps:

a) generating a positioning guide body portion according to the STL data;

b) designing the fixing nail sleeve buckle 5 and tongue side locating plane 6 matched with the outer surface of the fixing nail sleeve 2;

c) the positioning guide plate main body part and fixing nail sleeve buckle 5 and tongue side locating plane 6 are connected as a whole, generating the guide plate 4 (as shown in FIG. 3-1, 3-2).

8. The planting guide plate 7 is generated by the following steps:

a) selecting suitable implant sleeve 8 according to the type of the implant, and additional upper connecting rod 9 the implant sleeve 8 is firmly connected;

b) the connecting rod 9, tongue side locating plane 6, fixing nail sleeve buckle 5 and implant sleeve 8 are connected as a whole, generating planting guide 7 (as shown in FIG. 4-1, 4-2).

9. The method for generating repair guide plate 10:

a) according to the oral cavity occlusion condition of the patient virtual teeth, obtaining initial prosthesis (i.e., repairing dentition);

b) selecting a suitable base according to the type and orientation of the selected implant, obtaining the final prosthesis (namely repairing dentition);

c) connecting the connecting rod 9, tongue side locating plane 6, fixing nail sleeve buckle 5 and final prosthesis (repairing tooth row) are connected together, generating repairing guide plate 10 (as shown in FIG. 5-1, 5-2).

10. The substrate guide plate 1, the guide plate 4, the planting guide plate 7 and repairing guide plate 10 generated in the step 6, 7, 8 and 9 are output and printed by the 3D.

11. Use of Compound Guide (Clinical Surgery):

a) the doctor in the patient mouth to test, the guide plate 4 and base plate 1 are connected together and worn in the patient mouth (as shown in FIGS. 6-1 to 6-4), after confirming position according to the fixing nail sleeve 2 on the fixing nail, taking the guide plate 4.

b) drawing teeth according to the planning before operation;

c) then according to the substrate guide 1 of the fixing nail sleeve 2 outer surface and locating plane 3 on the planting guide 7, the substrate guide plate 1 of the locating plane 3 with the planting guide 7 of the tongue side locating plane 6; after wearing the planting guide 7, implanting the implant (as shown in FIGS. 7-1 to 7-4) through the implant sleeve 8.

d) after implanting the implant, taking off the planting guide plate 7. then installing temporary abutment 12 on the implant, and according to the fixing nail sleeve 2 outer surface of the substrate guide plate 1 and positioning plane 3 wearing repairing guide plate 10, the substrate guide plate 1 of the locating plane 3 with the repairing guide plate 10 of tongue side locating plane 6 (as shown in FIGS. 8-1 to 8-4). After the repair guide 10 is worn, the temporary abutment 12 is placed around the temporary abutment 12 to bond the temporary abutment 12 to the repair guide.

e) after bonding, taking the temporary base 12 and repairing guide plate 10, grinding the repairing guide plate 10 on the connecting rod 9, tongue side locating plane 6 and fixing nail sleeve buckle 5, leaving the repairing body, and grinding the temporary base 12 protruding the repairing guide plate 10 upper end.

f) removing the substrate guide plate 1; wearing the prosthesis in the step e); fixing the temporary base station 12 and implant.

### EXAMPLE 4

A composite guide plate, belonging to the mucosal support type composite guide plate, wherein the preparation and use (using the non-flap-free tooth-jaw case) process is as follows (as shown in FIG. 1-1):

1) on the oral mucosa, at least 4 point bonding resistance material on the dental arch mucosa or/and on the dental dyke.

2nd, carrying out CBCT shooting; obtaining CBCT data with anti-reflection material information.

3rd, obtaining the oral surface STL data with impedance material information, the method comprising:

a) taking the oral cavity impression; the blocking material will leave the mark on the corresponding position of the impression material; after the mould is hard to be printed, copying the blocking material to the impression; using the oral surface scanning device to scan the oral impression; obtaining the STL data with the information of the blocking material;

b) or using oral surface scanning device to directly scan in the oral cavity; obtaining STL data with anti-reflection material information;

c) or bonding the barrier material at the thickest and thinnest part of the oral mucosa, then displaying the bone in the CBCT data according to CBCT; tooth information and suspended resistance material information; because the suspension of the impedance material is adhered on the soft tissue, by analyzing the impedance material and bone relation, it can analyze the relationship and thickness of the bone and mucosa, so as to obtain soft tissue information, finally obtaining STL data.

4. matching the CBCT data with the STL data: integrating the CBCT data and STL data, taking the information of the blocking material as the matching basis, by adjusting the tooth tissue information in the CBCT data and STL data, finally matching the jaw bone information, tooth tissue information, soft tissue information.

5. Planning and retention of the implants and retention pins: according to the CBCT data and STL data matching information, obtaining the pre-tooth position and jaw bone condition, then according to the pre-tooth position and jaw bone condition, planning implant and fixing nail position.

The substrate guide plate 1 is formed by the following steps: designing the fixing nail sleeve 2 and locating plane 3, the combination is a whole, to obtain the fixing nail sleeve 2 of the substrate guide plate 1 (as shown in FIGS. 2-1 to 2-4).

7. generating the planting guide plate 7:

a) selecting a suitable implant sleeve 8 according to the type of the implant, and attaching the upper connecting rod 9 to each implant sleeve 8 is firmly connected.

b) the connecting rod 9, tongue side locating plane 6, fixing nail sleeve buckle 5 and implant sleeve 8 are connected as a whole, generating planting guide 7 (as shown in FIG. 4-1, 4-2).

8. The production of repair guide plate 10:

a) according to the oral cavity occlusion condition of the patient virtual teeth, obtaining initial prosthesis (i.e., repairing dentition);

b) selecting a suitable base according to the type and orientation of the selected implant, obtaining the final prosthesis (namely repairing dentition);

c) connecting the connecting rod 9, tongue side locating plane 6, fixing nail sleeve buckle 5 and final prosthesis (repairing tooth row) are connected together, generating repairing guide plate 10 (as shown in FIG. 5-1, 5-2).

9. The substrate guide plate 1 generated in step 6, 7, 8, planting guide plate 7 and repairing guide plate 10 output and performing the 3D printing.

10. Use of Compound Guide (Clinical Surgery):

a) the doctor in the patient mouth after wearing, wearing the substrate guide 1, confirming the position according to the fixing nail sleeve 2 on the fixing nail.

b) then according to the substrate guide 1 of the fixing nail sleeve 2 outer surface and locating plane 3 on the planting guide 7, the substrate guide 1 of the locating plane 3 with the planting guide 7 of tongue side locating plane 6; after wearing the planting guide 7, implanting the implant (as shown in FIGS. 7-1 to 7-4) through the implant sleeve 8.

c) after implanting the implant, taking off the planting guide plate 7. then installing temporary abutment 12 on the implant, and according to the fixing nail sleeve 2 outer surface of the substrate guide plate 1 and positioning plane 3 wearing repairing guide plate 10, the substrate guide plate 1 of the locating plane 3 with the repairing guide plate 10 of tongue side locating plane 6 (as shown in FIGS. 8-1 to 8-4). After the repair guide 10 is worn, the temporary abutment 12 is placed around the temporary abutment 12 to bond the temporary abutment 12 to the repair guide.

d) after bonding, taking the temporary base 12 and repairing guide plate 10, grinding the repairing guide plate 10 on the connecting rod 9, tongue side locating plane 6 and fixing nail sleeve buckle 5, leaving the prosthesis, and grinding the temporary base 12 protruding the upper end of the repairing guide plate 10.

e) taking off the substrate guide plate 1, d) in the step of repairing body, the temporary base 12 and implant is fixed.

### EXAMPLE 5

A composite guide plate, belonging to the bone support type composite guide plate, preparation and use (suitable for the need of osteotomy case) process (as shown in FIG. 1-2):

The position of the implant and retention pin is as follows: obtaining the pre-tooth position and jaw bone condition according to the obtained CBCT data; then according to the pre-tooth position and jaw bone condition, planning the implant and position of the fixing nail.

2. generating substrate guide plate 1: designing the fixing nail sleeve 2 and locating plane 3, the combination is a whole, to obtain the fixing nail sleeve 2 of the substrate guide plate 1 (as shown in FIGS. 2-1 to 2-4).

3rd, generating planting guide plate 7:

a) selecting suitable implant sleeve 8 according to the type of the implant, and additional upper connecting rod 9 the implant sleeve 8 is firmly connected;

b) the connecting rod 9, tongue side locating plane 6, fixing nail sleeve buckle 5 and implant sleeve 8 are connected as a whole, generating planting guide 7 (as shown in FIG. 4-1, 4-2).

4. The method for generating repair guide plate 10

a) according to the oral cavity occlusion condition of the patient virtual teeth, obtaining initial prosthesis (i.e., repairing dentition);

b) selecting a suitable base according to the type and orientation of the selected implant, obtaining the final prosthesis (namely repairing dentition);

c) connecting the connecting rod 9, tongue side locating plane 6, fixing nail sleeve buckle 5 and final prosthesis (repairing tooth row) are connected together, generating repairing guide plate 10 (as shown in FIG. 5-1, 5-2).

5th, the substrate guide plate 1 generated in the step 2, 3, 4, planting guide plate 7 and repairing guide plate 10 output and performing the 3D printing.

6. Use of Compound Guide (Clinical Surgery):

a) the doctor to flap in the patient mouth, then wearing the substrate guide 1, confirming the position according to the fixing nail sleeve 2 fixing nail, and then reference plane 3 the jaw bone 11 operation (as shown in FIG. 9).

b) then according to the substrate guide 1 of the fixing nail sleeve 2 outer surface and locating plane 3 on the planting guide 7, the substrate guide 1 of the locating plane 3 with the planting guide 7 of tongue side locating plane 6; after wearing the planting guide 7, implanting the implant (as shown in FIGS. 10-1 to 10-4) through the implant sleeve 8.

c) after implanting the implant, taking off the planting guide plate 7. then installing temporary abutment 12 on the implant, and according to the fixing nail sleeve 2 outer surface of the substrate guide plate 1 and positioning plane 3 wearing repairing guide plate 10, the substrate guide plate 1 of the locating plane 3 with the repairing guide plate 10 of tongue side locating plane 6 (as shown in FIGS. 11-1 to 11-4). After the repair guide 10 is worn, the temporary abutment 12 is placed around the temporary abutment 12 to bond the temporary abutment 12 to the repair guide.

d) after bonding, taking the temporary base 12 and repairing guide plate 10, grinding the repairing guide plate 10 on the connecting rod 9, tongue side locating plane 6 and fixing nail sleeve buckle 5, leaving the prosthesis, and grinding the temporary base 12 protruding the upper end of the repairing guide plate 10.

e) taking off the substrate guide plate 1; suturing the patient mucosa; wearing the prosthesis in the step d); fixing the temporary base station 12 and implant.

Although the embodiment of the utility model has been disclosed above, but it is not limited to the application in the specification and implementation manner. It can be applied to all kinds of fields suitable for the utility model. Additional modifications may be readily achieved for those skilled in the art. Therefore, without departing from the general concept as defined in the claims and equivalent range, the utility model is not limited to specific details and shown and described herein.

1. A composite guide plate, comprising:
a substrate guide plate (1) and a planting guide plate (7);
or substrate guide plate (1) and repairing guide plate (10);
or substrate guide plate (1), planting guide plate (7) and repairing guide plate (10);
a fixing nail sleeve (2) is arranged on the substrate guide plate (1);
an implant sleeve (8) and a fixing nail sleeve buckle (5) are arranged on the planting guide plate (7);
the fixing nail sleeve buckle (5) is buckled on the fixing nail sleeve (2) so that the planting guide plate (7) is connected with the substrate guide plate (1);
a fixing nail sleeve buckle (5) is arranged on the repairing guide plate (10);
the fixing nail sleeve buckle (5) is buckled on the fixing nail sleeve (2) so that the repairing guide plate (10) is connected with the substrate guide plate (1).

2. The composite guide plate according to claim 1, wherein the substrate guide plate (1) is further provided with a locating plane (3);
the planting guide plate (7) is further provided with a tongue side locating plane (6);
the tongue side locating plane (6) is matched with the locating plane (3) of the substrate guide plate (1);
the repairing guide plate (10) is further provided with a tongue side locating plane (6);
the tongue side locating plane (6) is matched with the locating plane (3) of the substrate guide plate (1).

3. The composite guide plate according to claim 2, further comprising:
a locating guide plate (4), the locating guide plate (4) is provided with a fixing nail sleeve buckle (5) and a tongue side locating plane (6);
the tongue side locating plane (6) is matched with the locating plane (3) of the substrate guide plate;
the fixing nail sleeve buckle (5) is buckled on the fixing nail sleeve (2) so that the locating guide plate (4) is connected with the substrate guide plate (1).

4. The composite guide plate according to claim 3, wherein the planting guide plate (7);
the repairing guide plate (10) and locating guide plate (4) are respectively provided with a connecting rod (9);
the connecting rod (9) is connected with the fixing nail sleeve buckle (5).

5. The composite guide plate according to claim 2, wherein the repairing guide plate (10) is provided with a repairing body;
the repairing body is connected with the tongue side locating plane (6) and fixing nail sleeve buckle (5).

6. The composite guide plate according to claim 5, wherein when the repairing body is finally planted, grinding the tongue side locating plane (6), fixing nail sleeve buckle (5) and connecting rod (9).

7. The composite guide plate according to claim 1, further comprising:
an implant, implanting the implant through the implant sleeve (8), the implant is connected with a temporary abutment (12).

8. The composite guide plate according to claim 7, wherein the temporary abutment (12) is bonded with the repairing body on the repairing guide plate (10).

9. The composite guide plate according to claim 1, wherein the inner surface of the fixing nail sleeve buckle (5) is jointed with the outer surface of the fixing nail sleeve (2);
the radian of the fixing nail sleeve buckle (5) is 180 degrees to −240 degrees;
the outer surface of the fixing nail sleeve buckle (5) is a concave-convex structure, an embedding structure, a square and a rectangular structure, a hexagonal structure, a rhombic structure or a circular arc structure.

10. The composite guide plate according to claim 1, wherein the substrate guide plate (1), planting guide plate (7), repairing guide plate (10) and a locating guide plate (4) are made of resin.
